(19) United States
(12) Patent Application Publication
(10) Pub. No.: US 2025/0380929 A1
(43) Pub. Date: Dec. 18, 2025

(54) GENERATING DATA SETS FOR MACHINE LEARNING USING ULTRASOUND IMAGING

(71) Applicant: ACOUSTIIC INC., Bellevue, WA (US)

(72) Inventors: Holly Lay, Seattle, WA (US); Paul Reynolds, Renton, WA (US)

(73) Assignee: ACOUSTIIC INC., Bellevue, WA (US)

(21) Appl. No.: 18/741,324

(22) Filed: Jun. 12, 2024

(51) Int. Cl. A61B 8/08; A61B 8/00

(52) U.S. Cl. CPC A61B 8/483; A61B 8/4488

(57) ABSTRACT

Systems and techniques are provided for generating data sets for machine learning using ultrasound imaging. Acoustic beams may be directed at a target with either or both of a first transducer array and a second transducer array of an ultrasound system. The second transducer array may receive reflected ultrasound that results from reflections of the one or more acoustic beams. The second transducer array may generate data based on the receiving of the reflected ultrasound. A computing and imaging device of the ultrasound system may generate material property data based on the data generated by the second transducer array. The computing and imaging device may store the material property data in a data set. A trainer may train a machine learning model using a portion of the data set as training data. The machine learning model may be used to generate adjustments to acoustic beams.

### BACKGROUND

Therapies using Focused ultrasound (FUS) may use Magnetic Resonance Imaging (MRI) to monitor the status of the patient and treatment progress via thermal dose calculation, tissue stiffness and change, and functional alteration (fMRI). MRI installations may have limited availability and be costly. This may limit the instances of therapies that use FUS that can be monitored using MRI. Other forms of monitoring that may be used during FUS treatment may include physiological feedback, advance simulation or other biological signal monitoring, such as EEG, EMG, EKG. These forms of monitoring may be second or third order, which may increase the risk of over-treatment or bad targeting.

### BRIEF DESCRIPTION OF THE DRAWINGS

The accompanying drawings, which are included to provide a further understanding of the disclosed subject matter, are incorporated in and constitute a part of this specification. The drawings also illustrate implementations of the disclosed subject matter and together with the detailed description serve to explain the principles of implementations of the disclosed subject matter. No attempt is made to show structural details in more detail than may be necessary for a fundamental understanding of the disclosed subject matter and various ways in which it may be practiced.

FIG. 1 shows an example system for generating data sets for machine learning using ultrasound imaging according to an implementation of the disclosed subject matter.

FIG. 2 shows an example arrangement for generating data sets for machine learning using ultrasound imaging according to an implementation of the disclosed subject matter.

FIG. 3 shows an example arrangement for generating data sets for machine learning using ultrasound imaging according to an implementation of the disclosed subject matter.

FIG. 4 shows an example arrangement for generating data sets for machine learning using ultrasound imaging according to an implementation of the disclosed subject matter.

FIG. 5 shows an example procedure for generating data sets for machine learning using ultrasound imaging according to an implementation of the disclosed subject matter.

FIG. 6 shows an example procedure for generating data sets for machine learning using ultrasound imaging according to an implementation of the disclosed subject matter.

FIG. 7 shows an example procedure for generating data sets for machine learning using ultrasound imaging according to an implementation of the disclosed subject matter.

FIG. 8 shows a computer according to an implementation of the disclosed subject matter.

FIG. 9 shows a network configuration according to an implementation of the disclosed subject matter.

### DETAILED DESCRIPTION

Ultrasound imaging may be used to monitor tissue changes in a target before, during and after FUS treatment. The ultrasound imaging may be performed using a monitoring system that is separate from or part of the same system as an ultrasound system used for FUS treatment. The ultrasound imaging may include doppler ultrasound, including color flow, power doppler and/or pulse wave doppler, harmonic imaging, shear wave imaging using acoustic radiation force or pulsative drive, and may use image post-processing for edge detection, speckle tracking, and spectral analysis, reflex transmission imaging for attenuation measurement, quantitative analysis for attenuation estimation and back-scatter calculation.

Ultrasound imaging may be used to determine absolute values or changes in the material properties of a target, such as tissue. The material properties determined using ultrasound imaging may include longitudinal and shear acoustic velocity, attenuation, thermal expansion coefficient, backscatter, average grain size, tissue nonlinearity, and flowrate for fluid. Ultrasound imaging may also be used to determine properties such as distortion or shifting of material around the target, for example, the patient's anatomy, during and after FUS treatment. The rates of change of the properties determined using ultrasound imaging may be cross-referenced with time, temperature, or other suitable factors. Ultrasound imaging may use multiple frequencies or broadband excitation to derive frequency dependence of the determined properties. The density of a target may be determined using computed tomography (CT) and used along with material properties of the target determined using ultrasound imaging to derive material acoustic impedance of the target. Absolute values for material properties may be determined using the data generated by ultrasound imaging along with other data that may be generated through MRI, CT, or other types of imaging, such as the location of a known marker/fiducial, common biological landmarks, or a through pitch/catch method with multiple ultrasound devices or multiple transducer elements within one physical package. The multiple ultrasound devices may be coaxial, coplanar, or non-coplanar depending on measurement methodology being used.

The determined material properties, and changes in those material properties, and anatomy may be used to assess therapy progression and effectiveness after FUS treatment or may be used to alter the FUS treatment as it is in progress and may also be used to assist diagnosis or detection of contraindications such as fibrosis or prior ablation. The determining of material properties and changes in material properties using ultrasound imaging may occur at any suitable time and interval, such as, for example, multiple times during FUS treatment, including as often as every therapy pulse. The determined material properties may also be used to adapt and improve the focus calculations, for example, for aberration correction, for the ultrasound used in FUS or other systems for either imaging or therapy. The determined material properties may also be used to assess changes in hydration or water content in targets, for example, tissues, for MRI image improvement or to allow correction of MRI images in the presence of EM aberration.

The determined material properties may be used to generate a data set. The data set may include material properties determined across any number of uses of FUS treatment. The data set may be used to create training data sets that may be used to train any suitable machine learning system using any suitable form of learning, such as, for example, neural networks of any suitable structure trained using backpropagation. The data set may be used to train machine learning systems to plan paths for acoustic beams used in FUS treatment and to monitor acoustic beam paths and overall treatment progress during FUS treatment. The use of ultrasound imaging during FUS treatment may allow for the collection of a large data set of material properties, which may then allow for the data set to be used as a training data set for the training of machine learning systems with minimal annotation to the data set, for example, minimal or no labeling by humans or labeling systems.

An ultrasound system for thermographic monitoring using ultrasound imaging may be implemented, for example, using a combined imaging/therapy transducer array, using multiple transducer arrays one of which is capable of transmitting ultrasound and another of which is capable of receiving ultrasound and which may be combined within a larger single device or in physically separated devices, or using an ultrasound imaging transducer and another method of generating ultrasound near a target such as photoacoustic generation or an implanted or ingested ultrasound transmitter.

The transducer arrays of the ultrasound system may include a single transducer element or may be multi-element, including single row, multi-row, or 2-D transducer arrays. The transducer arrays may be confocal or misaligned. The transducer elements used by the transducer arrays may be piezoelectric, electrostatic, electrostrictive, magnetostrictive, or photo-acoustic transducer elements and may be manufactured using bulk or micromachining techniques.

The transducer arrays of the ultrasound system may include built-in hardware and/or software feedback loop that may allow for the observation of the imaging output and modification of the treatment process in real time. A joint control system of the ultrasound system may control transducer arrays used for imaging and transducer arrays used for therapy with feedback either at a hardware or software level. The join control system may, for example, control mechanical motion of the imaging transducer array, steering of the acoustic beam generated by the imaging transducer array, and pulse and imaging mode changes for the imaging transducer array.

The material properties of a target, such as tissue, determined using ultrasound imaging may be used alone or with other data as a diagnostic tool or in real time monitoring of therapy being performed using the ultrasound system. The data for material properties stored in large data sets, for example, data sets of determined material properties of tissues of multiple patients, may be collated to estimate the properties of a target, for example, tissue, in patients depending upon the properties of the patients such as, for example, age, gender, hydration, and race. These estimated properties of the target may be used in therapeutic planning when positional data when other forms of imaging such as MRI or CT are not available and may be used to improve the performance of ultrasound systems when ultrasound alone is used for treatment.

The data for material properties in large data sets may also be used to generate maps of the target, for example, tissue maps, which may be used in a diagnostic manner. The data for material properties in large data sets may be used in conjunction with ultrasound data alone to accurately estimate the tissue properties without using additional forms of imaging, such as MR or CT, or with smaller and/or fewer ultrasound devices. Tissue maps may be used in therapy planning to optimize treatment plans. The tissue map for a particular patient may be generated once and used to plan FUS treatment or may be generated repeatedly during FUS treatment and used to adjust treatment as it occurs, by, for example, adjusting an acoustic beam generated by a therapy transducer array and used for treatment. The maps may be, for example, voxel-based volumetric maps of attenuation, longitudinal or shear velocity, nonlinearity, or other related material properties, that may be generated by correlating velocity and attenuation to get volumetric data on speed and velocity in a target and by using elastography. Paths for the acoustic beam used for therapy may be determined based on the volumetric maps.

The transducer arrays used for ultrasound imaging may operate in a pulse echo manner, where the same transducer array both sends an ultrasound pulse and receives the reflected ultrasound resulting from the ultrasound pulse, or a pitch catch manner, where one of the transducer arrays sends the ultrasound pulse and another transducer array receives the reflected ultrasound resulting from the ultrasound pules. The transducer arrays may use matrix capture by transmitting ultrasound on a single transducer element or group of transducer elements at one time and receiving reflected ultrasound on all transducer elements and repeating this until all transducer elements or groups of transducer elements have transmitted, plane wave capture by transmitting ultrasound in a single broad wave at multiple angles and then receiving the reflected ultrasound, or any other form of ultrasound imaging such as, for example, doppler imaging or harmonic imaging.

Ultrasound imaging may be used to determine the specific heat capacity of a target, for example, tissue, based on energy deposition of ultrasound energy within tissue and a known temperature of the target from ultrasound or MR thermography or direct measurement.

The absolute or changing values of the material properties of a target, such as tissue, as determined through ultrasound imaging, may be used to monitor ultrasound therapy, for example FUS treatment. Detecting changes in the material properties of a target using ultrasound imaging when different types and levels of excitation, for example, different levels and patterns of ultrasound, are applied to the target may be used in diagnosis of the target.

Ultrasound imaging may be used with or without the presence of contrast agents to enhance the imaging or to enhance nonlinearity or other ultrasound imaging factors.

Diagnostic data may also be obtained based on inter-relationships between different determined and derived mechanical properties of a target.

Data on ultrasound velocities may be used to improve ultrasound imaging by either the removal or reduction of assumptions of velocity used in standard ultrasound imaging, to improve the focusing of acoustic beams used for therapy, including reduction of energy deposition in unwanted areas, and to improve MR images by improved estimation of important properties of a target, such as the water content of tissue.

Values of mechanical stiffness may be determined from material properties such as velocity and density and may be used to determine the timing and/or frequency content of the acoustic beam used to apply ultrasound to a target and amplitudes that may cause cavitation for histotripsy at desired locations, for example, within or near the target, while ensuring sub-cavitation in sensitive regions that may be located near the target does not occur. Any material properties determined using ultrasound imaging may be used to minimize the amount of applied energy for therapeutic response for various types of treatment, including intrinsic or boiling histotripsy, thermal ablation, hypothermia, drug activation or enhancement, blood-brain barrier (BBB) opening, and neuromodulation, while meeting ALARA (As Low as Reasonably Achievable) medical norms. For example, using the material properties to more precisely determine material stiffness of target tissue may allow for the determination of that tissue's susceptibility to destruction at a specific pressure during histotripsy. This may allow for the determination of the minimum amplitude of the acoustic beam used for therapy that may damage that target tissue and no other tissue.

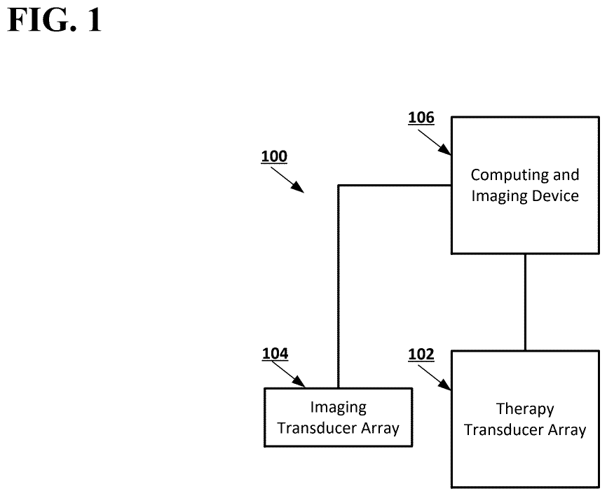

FIG. 1 shows an example arrangement for generating data sets for machine learning using ultrasound imaging according to an implementation of the disclosed subject matter. An ultrasound system 100 may include a therapy transducer array 102, an imaging transducer array 104, and a computing and imaging device 106. The therapy transducer array 102 may be an array of any suitable size, with any suitable number of transducer elements that may generate an acoustic beam that may be ultrasonic and may be used for therapy such as FUS treatment. The imaging transducer array 104 may be an array of any number of transducer elements that may be used for ultrasound imaging, and may be implemented using any number of physically separate transducer arrays. The therapy transducer array 102 and the imaging transducer array 104 may be connected to the computing and imaging device 106 through any suitable wired and/or wireless connections.

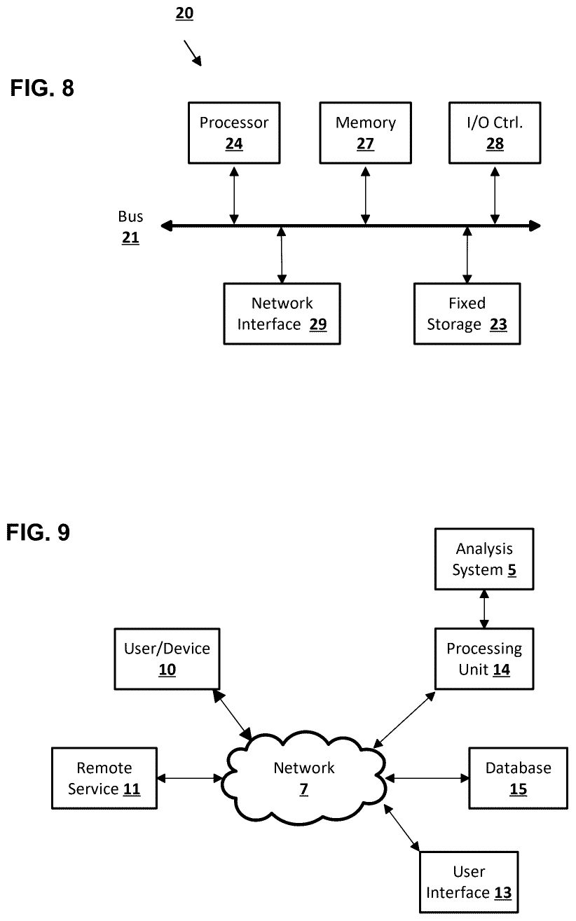

The computing and imaging device 106 may include any suitable computing hardware, running any suitable software, and any other suitable electronics to operate the ultrasound system 100, including supplying power and control signals to transducer elements of the therapy transducer array 102 and the imaging transducer array 104, receiving signals from the transducer elements of the therapy transducer array 102 and the imaging transducer array 104, performing any suitable computation to generate images from the signals received from the transducer elements of the therapy transducer array 102 and the imaging transducer array 104, and displaying generated images, for example, on a display directly connected to the computing and imaging device 106, or otherwise sending the generated images to a device, for example, a tablet or phone, that can display the generated images. The computing and imaging device 106 may use data from the imaging transducer array 104 to perform ultrasound imaging and determine material properties of a target, such as tissue within a patient, imaged by the imaging transducer array 104. The material properties of the target may be determined at any suitable time, including before, during, and after an acoustic beam generated by the therapy transducer array 102 is applied to the target. The computing and imaging device 106 may store the material properties determined for a target in a data set. The data set may be used in any suitable manner, including, for example, to generate training data sets that may be used to train machine learning systems. The computing and imaging device 106 may have any suitable interface to allow a user to control the ultrasound system 100. The computing and imaging device 106 may be or include a computer 20 as shown in in FIG. 8. The computing and imaging device 106 may also include any suitable electric and electronic components for delivering power to the therapy transducer array 102 and the imaging transducer array 104.

The therapy transducer array 102 and the imaging transducer array 104 may be implemented using a combined imaging/therapy transducer array, using multiple transducer arrays one of which is capable of transmitting ultrasound and another of which is capable of receiving ultrasound and which may be combined within a larger single device or in physically separated devices, or using an ultrasound imaging transducer and another method of generating ultrasound near a target such as photoacoustic generation or an implanted or ingested ultrasound transmitter.

The therapy transducer array 102 and the imaging transducer array 104 may include a single transducer element or may be multi-element, including single row, multi-row, or 2-D transducer arrays. The therapy transducer array 102 and the imaging transducer array 104 may be confocal or misaligned. The therapy array 102 may be able to use all or a subset of its transducer elements for imaging. The transducer elements used by therapy transducer array 102 and the imaging transducer array 104 may be piezoelectric, electrostatic, electrostrictive, magnetostrictive, or photo-acoustic transducer elements. The transducer elements may be manufactured using bulk or micromachining techniques.

The therapy transducer array 102 and the imaging transducer array 104 may include built-in hardware and/or software feedback loop that may allow for the observation of the imaging output and modification of the treatment process in real time. Imaging focus, mode, type, power, and other characteristics of the treatment process may be adjusted with therapy target location. A joint control system of the ultrasound system 100 may control the therapy transducer array 102 and the imaging transducer array 104 with feedback either at a hardware or software level. The joint control system may, for example, be a component of the computing and imaging device 106.

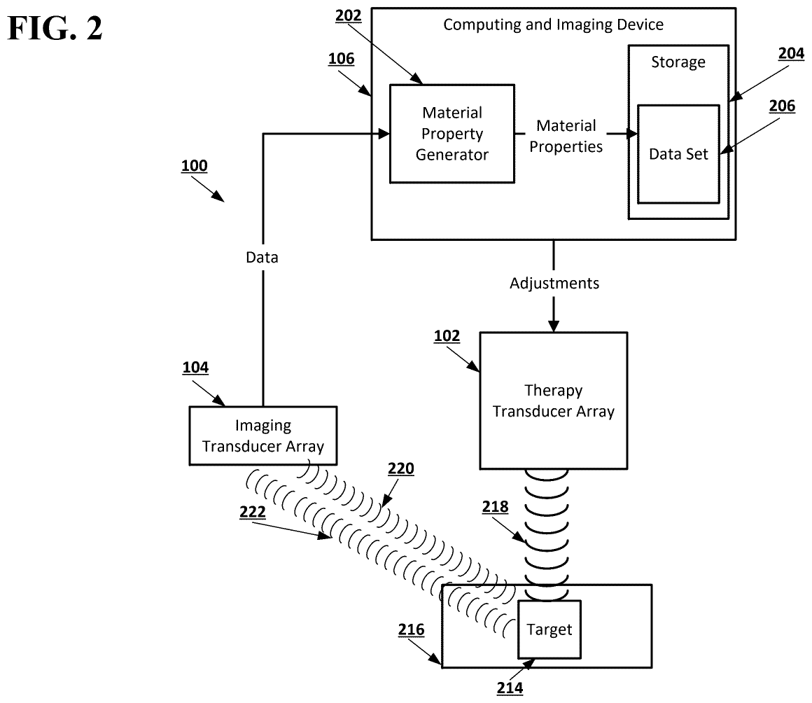

FIG. 2 shows an example arrangement for generating data sets for machine learning using ultrasound imaging according to an implementation of the disclosed subject matter. The ultrasound system 100, including the imaging transducer array 104 and a therapy transducer array 102 may be used to locate a target 214, for example, tissue, within a region of interest of a volume 216, such as a patient, using the imaging transducer array 104, and apply therapy treatment using an acoustic beam 218 generated by the therapy transducer array 102. The imaging transducer array 104 may image the area of the target 214 by receiving reflected ultrasound 222, which may result from the reflecting of the acoustic beam 218 and/or an acoustic beam 220 generated by the imaging transducer array 104, or any other ultrasonic acoustic beam generated and directed at the target 214 by any other suitable ultrasonic device.

The imaging transducer array 104 may generate data from the reflected ultrasound 222 and send the generated data to the computing and imaging device 106. The data may include any signals generated by the transducer elements of the imaging transducer array 104 and data generated by pre-processing the signals using any suitable hardware and software of the imaging transducer array 104.

The computing and imaging device 106 may process the received data from the imaging transducer array 104 with a material property generator 202. The material property generator 202 may be any suitable hardware and software of the computing and imaging device 106, or of another computing device in communication with the computing and imaging device 106, that may generate material property data using data received from the transducer array such as the imaging transducer array 104. The material property generator 202 may determine material property data for the target 214, and area near the target 214 around the volume 216, that is imaged by the imaging transducer array 104. The material properties that the material property generator 202 determines may include, for example, absolute values or changes in the material properties of a target 214, including longitudinal and shear acoustic velocity, attenuation, thermal expansion coefficient, backscatter, average grain size, tissue nonlinearity, and flowrate for fluid, along with properties such as distortion or shifting of material around the target 214, for example, the patient's anatomy, during and after FUS treatment. Data gathered from the imaging transducer array 104 over time may be used by the material property generator 202 to determine rates of change of the material properties cross-referenced with time, temperature, or other suitable factors. The acoustic beam 218 and/or the acoustic beam 220 may be generated using multiple frequencies or broadband excitation to allow for the derivation of frequency dependence of the determined material properties. The material property generator 202 may determine the density of the target 214 by using data from computed tomography (CT) along with the material properties of the target 214 to derive material acoustic impedance of the target 214. Absolute values for material properties may be determined using the data generated by imaging transducer array 104 along with other data that may be generated through MRI, CT, or other types of imaging, such as location of a known marker/fiducial or a through pitch/catch method with multiple ultrasound devices or multiple transducer elements within one physical package. The multiple ultrasound devices may be coaxial, coplanar, or non-coplanar depending on measurement methodology being used.

The determined material properties generated by the material property generator 202 may be used to generate a data set 206. The data set 206 may include material properties determined across any number of ultrasound imaging sessions, including those performed using the imaging transducer array 104 before, during, and/or after treatment, such as FUS treatment, performed by the therapy transducer array 102 and those performed by other imaging transducer arrays. Material properties determined from data generated by other imaging transducer arrays may be added to the data set 206 in any suitable manner. For example, the data set 206, or a copy thereof, may be stored on a server system and may be accessible to the computing and imaging device 106. The data set 206 may be used to generate training data sets that may be used to train any suitable machine learning system using any suitable form of learning, such as, for example, neural networks of any suitable structure. The data set 206 may be used to train machine learning systems to plan paths for acoustic beams used in FUS treatment, such as the acoustic beam 218, and to monitor acoustic beam paths and overall treatment progress during FUS treatment. The use of ultrasound imaging by the imaging transducer array 104 and other imaging transducer arrays during FUS treatment may allow for the collection of a large data set of material properties, which may then allow for the data set 206 to be used as a training data set for the training of machine learning systems with minimal annotation to the data set, for example, minimal or no labeling by humans or labeling systems.

The determined material properties generated by the material property generator 202, and changes in those material properties, and anatomy may be used to assess therapy progression and effectiveness after FUS treatment or may be used to alter the FUS treatment as it is in progress and may also be used to assist diagnosis or detection of contraindications such as fibrosis or prior ablation. The determining of material properties and changes in material properties using ultrasound imaging may occur at any suitable time and intervals, such as, for example, multiple times during FUS treatment, including as often as every therapy pulse. The determined material properties may also be used to adapt and improve the focus calculations for the ultrasound used in FUS or other systems for either imaging or therapy. The determined material properties may also be used to assess changes in hydration or water content in targets, for example, tissues, for MRI image improvement or to allow correction of MRI images in the presence of EM aberration. The computing and imaging device 106 may, for example, use the determined material properties to determine adjustments for the therapy transducer array 102 that may result in any suitable changes to the acoustic beam 218.

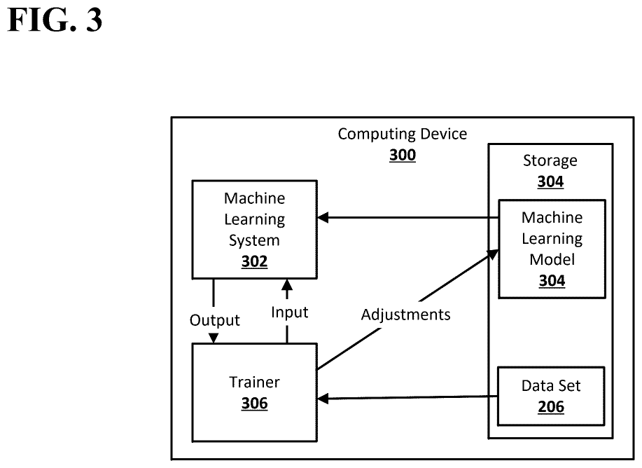

FIG. 3 shows an example arrangement for generating data sets for machine learning for thermographic monitoring using ultrasound imaging according to an implementation of the disclosed subject matter. A computing device 300 may be any suitable computing device, such as, for example, a computer 20 as described in FIG. 8, or component thereof. The computing device 300 may be a single computing device, or may include multiple connected computing devices, and may be, for example, a laptop, a desktop, an individual server, a server cluster, a server farm, or a distributed server system, or may be a virtual computing device or system, or any suitable combination of physical and virtual systems. The computing device 300 may be part of a computing system and network infrastructure or may be otherwise connected to the computing system and network infrastructure, including a larger server network which may include other server systems similar to the computing device 100. The computing device 300 may include any suitable combination of central processing units (CPUs), graphical processing units (GPUs), Field Programmable Gate Arrays (FPGA), tensor processing units (TPUs) or other Application Specific Integrated Circuits (ASICs). The computing device 300 may be, for example, the computing and imaging device 100 or a component thereof, or any other computing device.

A machine learning system 302 may be any suitable combination of hardware and software of the computing device 300 for any suitable machine learning system that may work with the machine learning model 304. The machine learning model 304 may, for example, be a neural network model of any suitable structure. The machine learning system 302 may use machine learning models, such as the machine learning model 304, to receive input and generate output. A trainer 306 may be any suitable combination of hardware and software of the computing device 300 that may work with the machine learning system 302 to train a machine learning model, such as the machine learning model 306. The trainer 306 may operate in any suitable manner, such as, for example, through backpropagation. The data set 206, or any subset of the data within the data set 206, may be used to generate a training data set to train the machine learning model 304 with the machine learning system 302 and the trainer 306 in any suitable manner. The machine learning model 304 may be trained to plan paths for acoustic beams, such as the acoustic beam 218, used in FUS treatment, adjustments to the properties of acoustic beams, and to monitor acoustic beam paths and overall treatment progress during FUS treatment. The trainer 306 may use data, for example, material properties gathered from a target, such as the target 214, using ultrasound imaging, from the data set 206 as input to the machine learning system 302 which may generate output using the machine learning model 304. The output of the machine learning system 302, for example, a path for an acoustic beam for FUS to be used on the target whose material properties were used as input to the machine learning model 302, may be sent to be evaluated by the trainer 306. The trainer 306 may evaluate the output for the machine learning system 302 in any suitable manner, for example, comparing the output path for the acoustic beam to a known correct path for the acoustic beam, and generate adjustments to the machine learning model 304 to determine how accurate the output is to the known correct path. The trainer 306 may apply the adjustments to the machine learning model 304, for example, adjusting weights of a neural network structure of the machine learning model 304. This may continue for as many training cycles as is suitable to result in a machine learning model 304 that generates accurate outputs when used with the machine learning system 302. The data set 206 may only need minimal human annotation in order to be used to generate training data sets to train the machine learning model 304.

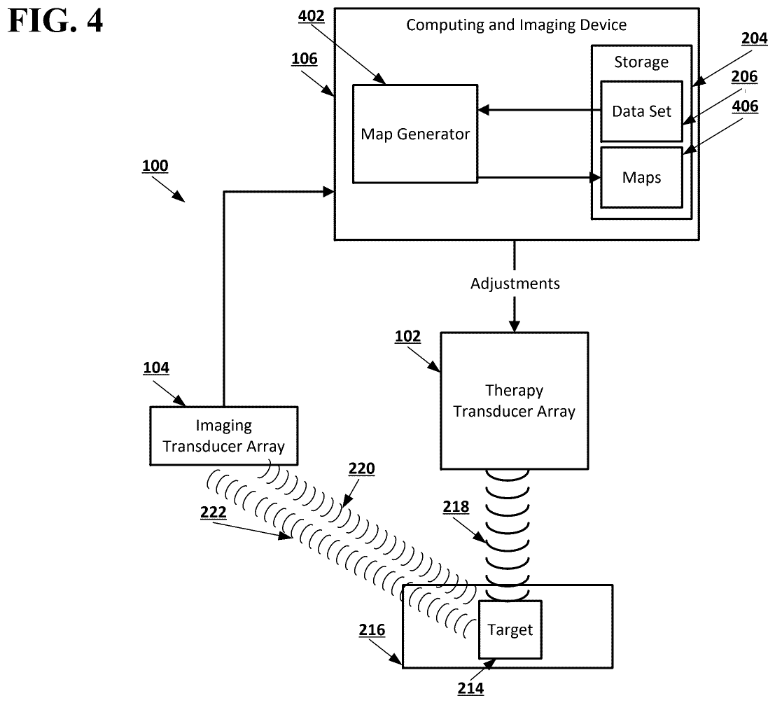

FIG. 4 shows an example arrangement for generating data sets for machine learning using ultrasound imaging according to an implementation of the disclosed subject matter. The computing and imaging device 106 may use the material properties stored in the data set 206 to generate maps with a map generator 402. The map generator 402 may be any suitable hardware and software of the computing and imaging device 106, or of another computing device in communication with the computing and imaging device 106, that may generate maps using material property data stored in the data set 206. The map generator 402 may generate maps of targets, such as the target 214, which may be used in a diagnostic manner. A map generated by the map generator 402 may be stored as maps 406 in the storage 204, or in any other suitable storage device. The maps 406 may be, for example, tissue maps that may be used in therapy planning to optimize treatment plans. The tissue map for a particular patient may be generated once from material property for that patient stored in the data set 206 and used to plan FUS treatment or may be generated repeatedly during FUS treatment and used to adjust treatment as it occurs, by, for example, adjusting the acoustic beam 218 generated by the therapy transducer array 102 and used for treatment. The maps 406 may be, for example, voxel-based volumetric maps of attenuation, longitudinal or shear velocity, or other related material properties, that may be generated by correlating velocity and attenuation to get volumetric data on speed and velocity in a target and by using elastography. Paths for the acoustic beam 218 used for therapy may be determined based on the volumetric maps 406.

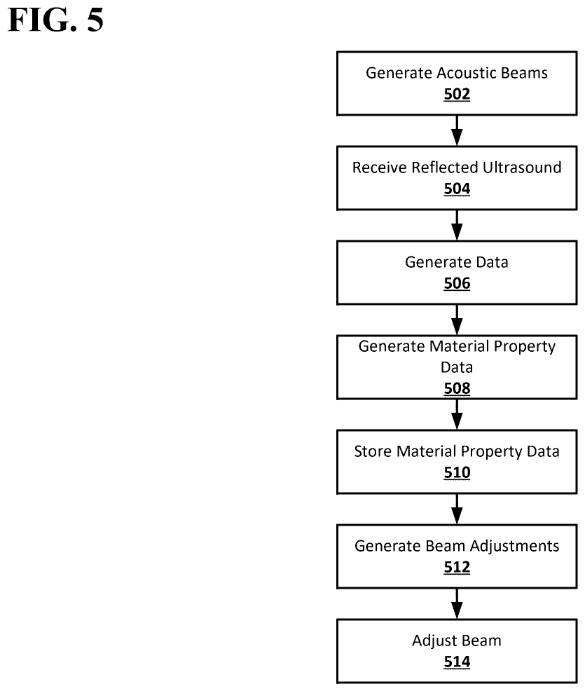

FIG. 5 shows an example arrangement for generating data sets for machine learning using ultrasound imaging according to an implementation of the disclosed subject matter. At 502, acoustic beams may be generated. For example, either or both of the therapy transducer array 102 and the imaging transducer array 104 may generate acoustic beams, for example, the acoustic beams 218 and 220, directed towards a location of the target 214 in the volume 216.

At 504, reflected ultrasound may be received. For example, any of the acoustic beams 218 and 220 may reflect off of material of the volume 216 and the target 214, resulting in the reflected ultrasound 222 that may be received at the transducer elements of the imaging transducer array 104.

At 506 data may be generated. For example, the imaging transducer array 104 may generate data, for example, imaging data, based on the reflected ultrasound 222. The reflected ultrasound 222 may be received at transducer elements of the imaging transducer array 104 which may operate in a receiving mode and may generate signals based on the reflected ultrasound 222. The generated signals may be data, such as imaging data, doppler, shear wave or other echo data. The data may be used to generate images of the area within the fields of view of the imaging transducer array 104, which may, for example, include the target 214.

At 508, material property data may be generated. For example, the data may be sent from the imaging transducer array 104 to the computing and imaging device 106. Data from other sources, such as MR or CT, may also be sent to the computing and imaging device 106. The material property generator 202 of the computing and imaging device 106 may use the data from the imaging transducer 104 and from the other sources to generate material property data. The material property data may include material properties such as, for example, absolute values or changes in the material properties of a target 214, including longitudinal and shear acoustic velocity, attenuation, thermal expansion coefficient, backscatter, average grain size, tissue nonlinearity, and flowrate for fluid, along with properties such as distortion or shifting of material around the target 214, for example, the patient's anatomy, during and after FUS treatment. Data gathered from the imaging transducer array 104 over time may be used by the material property generator 202 to determine rates of change of the material properties cross-referenced with time, temperature, or other suitable factors. The acoustic beam 218 and/or the acoustic beam 220 may be generated using multiple frequencies or broadband excitation to allow for the derivation of frequency dependence of the determined material properties. The material property generator 202 may determine the density of the target 214 by using data from computed tomography (CT) along with the material properties of the target 214 to derive material acoustic impedance of the target 214. Absolute values for material properties may be determined using the data generated by imaging transducer array 104 along with other data that may be generated through MRI, CT, or other types of imaging, such as location of a known marker/fiducial or a through pitch/catch method with multiple ultrasound devices or multiple transducer elements within one physical package. The multiple ultrasound devices may be coaxial, coplanar, or non-coplanar depending on measurement methodology being used.

At 510, the material property data may be stored. For example, the determined material properties generated by the material property generator 202 may be used to generate, or may be added to, the data set 206 stored in the storage 204. The data set 206 may include material properties determined across any number of ultrasound imaging or therapy sessions, including those performed using the imaging transducer array 104 before, during, and/or after treatment, such as FUS treatment, performed by the therapy transducer array 102 and those performed by other imaging transducer arrays or other imaging modalities such as MR or CT. Material properties determined from data generated by other imaging transducer arrays may be added to the data set 206 in any suitable manner. For example, the data set 206, or a copy thereof, may be stored on a server system and may be accessible to the computing and imaging device 106.

At 512, beam adjustments may be generated. For example, the computing and imaging device 106 may generate adjustments for the acoustic beam 218 based on material properties of the target 214 as determined by the material property generator 202. The beam adjustments may be generated in any suitable manner, including, for example, by a machine learning system, such as the machine learning system 302, using a machine learning model, such as the machine learning model 304. The adjustments may be any suitable adjustments to the acoustic beam 218, including, for example, adjustments to the path, timing, and frequency content of the acoustic beam 218.

At 514, the beam may be adjusted. For example, the computing and imaging device 106 may implement any adjustments determined for the acoustic beam 218 through control signals sent to the therapy transducer array 102. The control signals may cause the therapy transducer array 102 to generate the acoustic beam 218 in a manner that is consistent with the determined adjustments. The imaging transducer array 104 may also alter its beam, for example, the acoustic beam 220, or imaging mode based on this data to improve or adjust imaging functionality and performance.

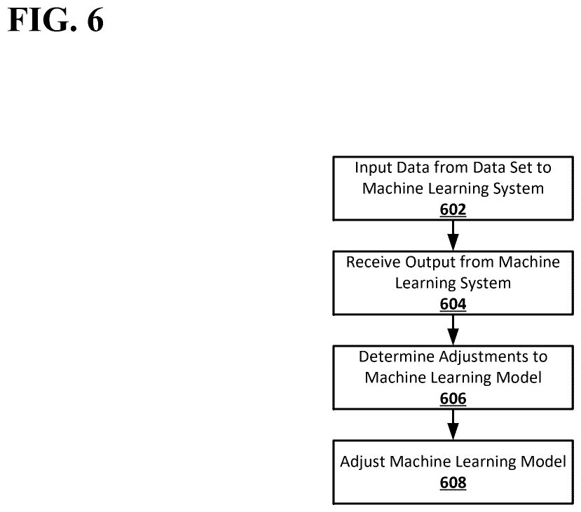

FIG. 6 shows an example arrangement for generating data sets for machine learning using ultrasound imaging according to an implementation of the disclosed subject matter. At 602, data from a data set may be input to a machine learning system. For example, the trainer 306 may input material property data from the data set 206 to the machine learning system 302. The data set 206 may be used to generate a training data set from which data may be input to the machine learning system 302. The material property data may include material properties determined for a single target, for example, the target 214. The material property data may be from any suitable temporal period over which the single target was imaged using ultrasound imaging by, for example, the imaging transducer array 104.

At 604, output may be received from the machine learning system. For example, the trainer 306 may receive output generated by the machine learning system 302, which may have used the machine learning model 304, which may be a neural network, to process the input material property data from the data set 206. The output may be, for example, a path for an acoustic beam such as the acoustic beam 218 to be used in treatment of the target 214 or may be an evaluation of treatment already applied to the target 214 using the acoustic beam 218.

At 606, adjustments to the machine learning model may be determined. For example, the trainer 306 may evaluate the output of the machine learning system 302 in any suitable manner. For example, the trainer 306 may evaluate the output received from the machine learning system 306 against a known desired output based on the material property data from the data set 206 that was input to the machine learning system 302 to determine the accuracy of the output of the machine learning system 302. The trainer 306 may determine adjustments to the machine learning model 304 based on the results of the evaluation of the output of the machine learning system 302. For example, the adjustments may be determined according to any suitable loss or training function.

At 608, the machine learning model may be adjusted. For example, the trainer 306 may adjust the machine learning model 304 based on the determined adjustments. The determined adjustments may be applied to the machine learning model 304 in any suitable manner, such as, for example, through backpropagation to adjust the weights of a neural network. The trainer 306 may repeat inputting data from the data set 206 to the machine learning system 302, receiving output from the machine learning system 302, determining adjustments to the machine learning model 304, and adjusting the machine learning model 304, for any suitable number of iterations over any suitable time period, including, for example, until output from the machine learning system 302 using the machine learning model 304 demonstrates a threshold level of accuracy, or continuously as new material property data is assed to the data set 206 from additional ultrasound imaging of additional targets.

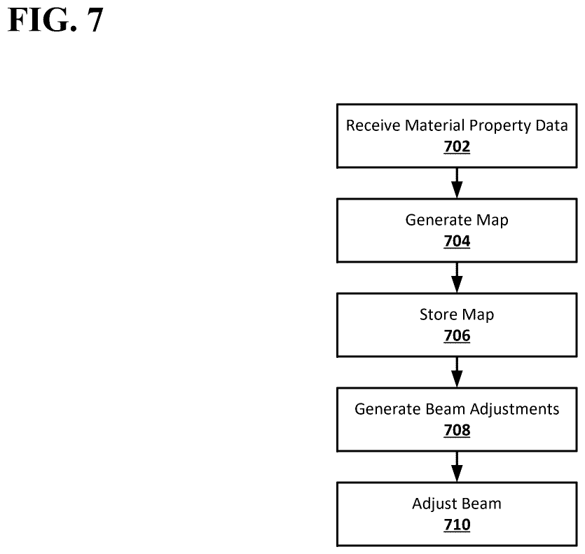

FIG. 7 shows an example arrangement for generating data sets for machine learning using ultrasound imaging according to an implementation of the disclosed subject matter. At 702, material property data may be received. For example, the map generator 402 may receive material property data from the data set 206. The material property data received by the map generator 402 may include material properties determined form data collected from ultrasound imaging of the target 214 before, during, or after application of the acoustic beam 218 to the target 214, for example, as a part of FUS treatment.

At 704, a map may be generated. For example, the map generator 402 may use the material property data to generate a map of the area from which the data used to generate the material property data was generated using ultrasound imaging. The map may include the target 214, or portions thereof, and portions of the volume 216 in the vicinity of the target 214. The map may be, for example, voxel-based volumetric maps of attenuation, longitudinal or shear velocity, or other related material properties, that may be generated by correlating velocity and attenuation to get volumetric data on speed and velocity in a target and by using elastography. Paths for the acoustic beam used for therapy or improved imaging may be determined based the volumetric maps.

At 706 the map may be stored. For example, the map generated by the map generator 402 may be stored with the maps 406 in the storage 204. The maps 406 may include maps generated from material property data determined across any number of ultrasound imaging sessions, including those performed using the imaging transducer array 104 before, during, and/or after treatment, such as FUS treatment, performed by the therapy transducer array 102 and those performed by other imaging transducer arrays or other imaging modalities such as MR or CT. Maps may be added to the maps 406 in any suitable manner. For example, the maps 406, or a copy thereof, may be stored on a server system and may be accessible to the computing and imaging device 106.

At 708, beam adjustments may be generated. For example, the computing and imaging device 106 may generate adjustments for the acoustic beam 218 based on maps generated by the map generator 402. The beam adjustments may be generated in any suitable manner, including, for example, by a machine learning system, such as the machine learning system 302, using a machine learning model, such as the machine learning model 304. The adjustments may be any suitable adjustments to the acoustic beam 218, including, for example, adjustments to the path, timing, and frequency content of the acoustic beam 218. The beam adjustments may be generated as close in time as possible to the generation of a map during treatment of the target 214, for example, during FUS treatment.

At 710, the beam may be adjusted. For example, the computing and imaging device 106 may implement any adjustments determined for the acoustic beam 218 through control signals sent to the therapy transducer array 102. The control signals may cause the therapy transducer array 102 to generate the acoustic beam 218 in a manner that is consistent with the determined adjustments. The imaging transducer array 104 may also alter its beam, for example, the acoustic beam 220, or imaging mode based on this data to improve or adjust imaging functionality and performance.

Implementations of the presently disclosed subject matter may be implemented in and used with a variety of component and network architectures. FIG. 8 is an example computer 20 suitable for implementations of the presently disclosed subject matter. The computer 20 includes a bus 21 which interconnects major components of the computer 20, such as a central processor 24, a memory 27 (typically RAM, but which may also include ROM, flash RAM, or the like), an input/output controller 28, a user display 22, such as a display screen via a display adapter, a user input interface 26, which may include one or more controllers and associated user input devices such as a keyboard, mouse, and the like, and may be closely coupled to the I/O controller 28, fixed storage 23, such as a hard drive, flash storage, Fibre Channel network, SAN device, SCSI device, and the like, and a removable media component 25 operative to control and receive an optical disk, flash drive, and the like.

The bus 21 allows data communication between the central processor 24 and the memory 27, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components. Applications resident with the computer 20 are generally stored on and accessed via a computer readable medium, such as a hard disk drive (e.g., fixed storage 23), an optical drive, floppy disk, or other storage medium 25.

The fixed storage 23 may be integral with the computer 20 or may be separate and accessed through other interfaces. A network interface 29 may provide a direct connection to a remote server via a telephone link, to the Internet via an internet service provider (ISP), or a direct connection to a remote server via a direct network link to the Internet via a POP (point of presence) or other technique. The network interface 29 may provide such connection using wireless techniques, including digital cellular telephone connection, Cellular Digital Packet Data (CDPD) connection, digital satellite data connection, or the like. For example, the network interface 29 may allow the computer to communicate with other computers via one or more local, wide-area, or other networks, as shown in FIG. 9.

Many other devices or components (not shown) may be connected in a similar manner (e.g., document scanners, digital cameras, and so on). Conversely, all of the components shown in FIG. 8 need not be present to practice the present disclosure. The components can be interconnected in different ways from that shown. The operation of a computer such as that shown in FIG. 8 is readily known in the art and is not discussed in detail in this application. Code to implement the present disclosure can be stored in computer-readable storage media such as one or more of the memory 27, fixed storage 23, removable media 25, or on a remote storage location.

FIG. 9 shows an example network arrangement according to an implementation of the disclosed subject matter. One or more clients 10, 11, such as local computers, smart phones, tablet computing devices, and the like may connect to other devices via one or more networks 7. The network may be a local network, wide-area network, the Internet, or any other suitable communication network or networks, and may be implemented on any suitable platform including wired and/or wireless networks. The clients may communicate with one or more servers 13 and/or databases 15. The devices may be directly accessible by the clients 10, 11, or one or more other devices may provide intermediary access such as where a server 13 provides access to resources stored in a database 15. The clients 10, 11 also may access remote platforms 17 or services provided by remote platforms 17 such as cloud computing arrangements and services. The remote platform 17 may include one or more servers 13 and/or databases 15.

More generally, various implementations of the presently disclosed subject matter may include or be implemented in the form of computer-implemented processes and apparatuses for practicing those processes. The disclosed subject matter also may be implemented in the form of a computer program product having computer program code containing instructions implemented in non-transitory and/or tangible media, such as floppy diskettes, CD-ROMs, hard drives, USB (universal serial bus) drives, or any other machine readable storage medium, wherein, when the computer program code is loaded into and executed by a computer, the computer becomes an apparatus for practicing implementations of the disclosed subject matter. Implementations also may be implemented in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing implementations of the disclosed subject matter. When implemented on a general-purpose microprocessor, the computer program code segments configure the microprocessor to create specific logic circuits. In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions.

Implementations may use hardware that includes a processor, such as a general-purpose microprocessor, Field Programmable Gate Array(s) (FPGAs) and/or one or multiple Application Specific Integrated Circuits (ASICs) that embodies all or part of the techniques according to embodiments of the disclosed subject matter in hardware and/or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The foregoing description, for purpose of explanation, has been described with reference to specific implementations. However, the illustrative discussions above are not intended to be exhaustive or to limit implementations of the disclosed subject matter to the precise forms disclosed. Many modifications and variations are possible in view of the above teachings. The implementations were chosen and described in order to explain the principles of implementations of the disclosed subject matter and their practical applications, to thereby enable others skilled in the art to utilize those implementations as well as various implementations with various modifications as may be suited to the particular use contemplated.

1. A system for ultrasound imaging comprising:
a first transducer array comprising transducer elements and configured to generate an acoustic beam for therapy;
a second transducer array comprising transducer elements and configured to receive reflected ultrasound and generate data based on the received reflected ultrasound;
a computing and imaging device connected to the first transducer array and the second transducer array and configured to receive the data generated by the second transducer array and generate material property data from the data received from the second transducer array.

2. The system of claim 1, wherein the material property data comprises one or more of absolute values or changes in longitudinal and shear acoustic velocity, attenuation, thermal expansion coefficient, backscatter, average grain size, tissue nonlinearity, flowrate for fluid, and distortion or shifting of material.

3. The system of claim 2, wherein the computing and imaging device is further configured to generate one or more maps based on the material property data.

4. The system of claim 3, wherein the computing and imaging device is further configured to generate based on the one or more maps one or more adjustments to one or both of the acoustic beam for therapy generated by the first transducer array and an acoustic beam generated by the second transducer array and to send control signals to one or both of the first transducer array and the second transducer array based on the adjustments.

5. The system of claim 1, wherein the computing and imaging device is configured to store the material property data in a data set.

6. The system of claim 5, further comprising a trainer configured to train a machine learning model using at least a portion of the data set as training data.

7. The system of claim 6, wherein the computing and imaging device is further configured to generate with a machine learning system and the machine learning model one or more adjustments to the acoustic beam for therapy generated by the first transducer array and to send control signals to the first transducer array based on the adjustments.

8. The system of claim 1, wherein the computing and imaging device is further configured to generate based on the material property data one or more adjustments to one or both of the acoustic beam for therapy generated by the first transducer array and an acoustic beam generated by the second transducer array and to send control signals to one or both of the first transducer array and the second transducer array based on the adjustments.

9. The system of claim 1, wherein the acoustic beam generated by the first transducer array is directed at a target comprising tissue.

10. The system of claim 9, wherein the material property data comprises material properties of the tissue.

11. The system of claim 1, wherein the computing device is further configured to:
determine, based on the material property data, a material stiffness of target tissue, and
determine, based on the material stiffness of the target tissue, minimum amplitudes to damage the target tissue without damaging other tissue that is not the target tissue.

12. The system of claim 1, wherein the first transducer array and the second transducer array are implemented as the same transducer array, or wherein the first transducer array is further and configured to receive reflected ultrasound and generate data based on the reflected ultrasound received at the first transducer array.

13. The system of claim 1, wherein the computing and imaging device is further configured to use data from Magnetic Resonance (MR) or Computed Tomography (CT) in addition to the data generated by the second transducer array to generate the material property data.

14. A method for ultrasound imaging comprising:
generating, one or more acoustic beams directed at a target with one or more of a first transducer array and a second transducer array of an ultrasound system;
receiving, at the second transducer array, reflected ultrasound that results from reflections of the one or more acoustic beams;
generating, by the second transducer array, data based on the receiving of the reflected ultrasound;
generating, by a computing and imaging device of the ultrasound system, material property data based on the data generated by the second transducer array; and
storing, by the computing and imaging device, the material property data in a data set.

15. The method of claim 14, wherein the material property data comprises one or more of absolute values or changes in longitudinal and shear acoustic velocity, attenuation, thermal expansion coefficient, backscatter, average grain size, tissue nonlinearity, flowrate for fluid, and distortion or shifting of material.

16. The method of claim 14, further comprising generating one or more maps based on the material property data.

17. The method of claim 16, further comprising generating, by the computing and imaging device, based on the one or more maps one or more adjustments to one or both of the acoustic beam for therapy generated by the first transducer array and an acoustic beam generated by the second transducer array and to send control signals to one or both of the first transducer array and the second transducer array based on the adjustments.

18. The method of claim 14, further comprising training a machine learning model using at least a portion of the data set as training data.

19. The method of claim 18, further comprising generating, by the computing and imaging device one or more adjustments to the acoustic beam for therapy generated by the first transducer array and to send control signals to the first transducer array based on the adjustments.

20. The method of claim 14, further comprising generating based on the material property data one or more adjustments to the acoustic beam for therapy generated by the first transducer array and to send control signals to the first transducer array based on the adjustments.

21. The method of claim 14, wherein the target comprises tissue of a patient.

22. The method of claim 18, wherein the material property data comprises material properties of the tissue.

23. The method of claim 14, wherein at least one of the one or more acoustic beams is an acoustic beam for focused ultrasound therapy.

24. The method of claim 14, further comprising:
determining, based on the material property data, a material stiffness of target tissue, and
determining, based on the material stiffness of the target tissue, minimum amplitudes to damage the target tissue without damaging other tissue that is not the target tissue.

25. The method of claim 14, wherein the first transducer array and the second transducer array are implemented as the same transducer array, and further comprising receiving, with the first transducer array reflected ultrasound and generating data based on the reflected ultrasound received at the first transducer array.

26. The method of claim 14, further comprising using, by the computing and imaging device, data from Magnetic Resonance (MR) or Computed Tomography (CT) in addition to the data generated by the second transducer array to generate the material property data.
